(19) United States
(12) Patent Application Publication
(10) Pub. No.: US 2018/0256021 A1
(43) Pub. Date: Sep. 13, 2018

(54) LASER SURGICAL INSTRUMENT FOR SPINAL ENDOSCOPIC DECOMPRESSION

(71) Applicant: Beth Israel Deaconess Medical Center, Boston, MA (US)

(72) Inventor: Jatinder S. Gill, Lincoln, MA (US)

(21) Appl. No.: 15/844,440

(22) Filed: Dec. 15, 2017

Related U.S. Application Data
(60) Provisional application No. 62/435,675, filed on Dec. 16, 2016.

(51) Int. Cl. A61B 1/313; A61B 1/32; A61B 18/24; A61B 1/00

(52) U.S. Cl. CPC A61B 1/3135; A61B 1/32; A61B 18/24; A61B 2018/0044; A61B 1/00082; A61B 1/00045; A61B 1/00066; A61B 1/00089

(57) ABSTRACT

The present invention relates to a flexible laser surgical instrument for endoscopic spinal decompression and methods thereof. Various methods of accessing the epidural space with this instrument are described. The instrument design enables placement of the device through several approaches. It is then advanced under fluoroscopic (X-Ray), for example, into areas of the spine including lumbar (low back), thoracic (mid and upper back) and cervical (neck). The pathologies encroaching upon the spinal space can then be visualized wherein the epidural membrane can optionally be displaced to further aid in visualization. Methods utilizing a CO₂ laser for laser ablation, for example, are employed for the removal of tissue pathologies within the epidural space.

### CROSS REFERENCE TO RELATED APPLICATION

This application claims the priority of U.S. Provisional Application No. 62/435,675 filed Dec. 16, 2016, the entire contents of the above application being incorporated herein by reference.

### BACKGROUND OF THE INVENTION

The epidural space encloses the spinal canal and is a common area of spine pathology such as disc herniation or spinal stenosis. At the current time open surgical approaches are the only reliable method to address these conditions in the spine. Given the invasive nature of the surgery, this remains a last resort and has long term deleterious consequences. The epidural space can be accessed with needles and catheters. The role of minimally invasive epidural surgery however remains limited. This is because of technological limitations to achieve safe, precise and adequate decompression, as the space is very small for large rigid scopes. Current minimally invasive solutions also lack advanced visualization capabilities to guide procedures.

Epiduroscopy may also be used in combination with a laser for ablating a disc where the resulting debris can be resorbed or manual removal of small disc fragments. The primary reason epiduroscopic surgery has not advanced involves the difficulties for visualizing the structures in the epidural space. Safety is also a concern because of collateral damage that could result in a sensitive nerve area or the possibility of damaging the wrong structure due to poor visibility.

Currently the only methods attempting visualization in the epidural space involve fluid distension, balloon neuroplasty, or a balloon cannula system. However, these methods are inadequate to support epiduroscopic surgery. It is clear from the above that there is an ongoing need for improvements in minimally invasive decompressive surgery of the spine.

### SUMMARY OF THE INVENTION

The present invention addresses the problems of conventional endoscopic spinal decompression surgery by providing a flexible imaging endoscope having a diameter of 5 mm or less that provides visualization and ablation of tissue associated with a herniated disc, for example, without damaging adjacent structures. More specifically encroaching structures in the epidural space such as a herniated disc, or ligament (as seen in spinal stenosis), and other encroaching structures can be safely removed in a minimally invasive manner using a laser instrument. Devices and methods of preferred embodiments are used to displace the epidural membrane to enable visualization and ablation of a structure intruding into the epidural space.

A preferred embodiment can employ a tubular body having a working channel extending from a proximal end to a distal end in which a fiber optic device can be inserted for delivering light having an energy density sufficient to ablate tissue. The tubular body can include device elements that distend the epidural diameter to provide improved visualization. By dilating the epidural space the user can more efficiently direct pulsed laser illumination onto tissues to be removed. A lens or lens system can be used on the distal end of the fiber optic device to form a beam of light having a desired shape at a selected distance at which the tissue to be ablated is located. A beam can have a selected spot size and energy distribution suitable to remove a selected volume of tissue in response to a pulse or sequence of pulses from the laser. Due to its emission wavelength, a CO₂ laser is preferably used for the tissue removal process, although other lasers emitting in the infrared or near infrared portion of the electromagnetic spectrum can also be used such as a Nd:YAG or Ho:YAG lasers emitting in the range of 1400 nm to 1908 nm, for example, or light emitting diode (LED) lasers. The waveguide used for delivery of CO₂ laser light can employ different distal beam shaping elements to precisely define the ablation volume for each light pulse.

A preferred embodiment uses an imaging device such as a CCD or CMOS digital imager to visualize the surgical region of interest. The imaging device preferably has at least 50,000 pixels, and preferably more than 1 million pixels for high resolution imaging at video frame rates. The imaging device can be mounted for positioning at the distal end of the device or a working channel within the device, or alternatively, can be optically coupled to a proximal end of a fiber optic imaging channel that can extend through the device or working channel to enable viewing of the region of interest. The imaging device can be mounted within a second tubular body in which laser light delivery system can also be mounted such that the imaging device and related optical elements are arranged to view the illuminated region of tissue. A second white light source such as one or more light emitting devices (LED) can be used to provide illumination of the small surgical field of view. The LEDs or fiber optic illumination elements can be arranged in an annular array at the distal end of the device to provide more uniform illumination.

### BRIEF DESCRIPTION OF THE DRAWINGS

FIG. 1 is an image depicting the structure of the human spine.

FIG. 2 schematically depicts the structure of an individual vertebra with the intervertebral disc on top.

FIG. 3 schematically depicts disc herniation.

FIG. 4 schematically depicts spinal stenosis.

FIG. 5 schematically depicts the caudal approach for epiduroscopy.

FIG. 6 schematically depicts the interlaminar approach for epiduroscopy.

FIG. 7 schematically depicts the transforaminal approach for epiduroscopy.

FIG. 8 schematically depicts the caudal, interlaminar, and transforaminal approaches for epiduroscopy.

FIG. 9 depicts a straight needle and a curved needle according to an embodiment.

FIG. 10 schematically depicts a wire according to multiple embodiments.

FIG. 11 schematically depicts a tip of an epiduroscope according to an embodiment.

FIGS. 12A-12B schematically depicts unstowing and stowing of the tip of the epiduroscope according to an embodiment.

FIGS. 13A-13B schematically depict unstowing and stowing of the tip of the epiduroscope when the tip is made of nitinol in an expanded shape according to an embodiment.

FIGS. 14A and 14B schematically depicts unstowing and stowing of the tip of the epiduroscope when the tip is unstowed with a balloon according to an embodiment.

FIG. 15 schematically depicts unstowing and stowing of the tip of the epiduroscope when a stowable sheath is behind and external to the tip according to an embodiment.

FIG. 16A schematically depicts a balloon distally advanced over a herniated disc according to an embodiment.

FIG. 16B schematically depicts a balloon proximally advanced over a herniated disc according to an embodiment.

FIG. 17A schematically depicts an epiduroscope moving between a membrane and a spinal bone according to an embodiment.

FIG. 17B schematically depicts an epiduroscope approaching an intruding pathology according to an embodiment.

FIG. 17C schematically depicts an epiduroscope with a hinged member according to an embodiment.

FIG. 17D schematically depicts a laser, lenses, and a digital imager of an epiduroscope according to an embodiment.

FIG. 17E schematically illustrates a cross sectional view of the device depicted in FIG. 17D.

FIG. 17F schematically depicts a laser, lenses, and a fiber optic imaging channel of an epiduroscope according to an embodiment.

FIGS. 17G and 17H illustrate preferred embodiments of a tissue ablation device in accordance with preferred embodiments of the invention.

FIG. 17I illustrates an embodiment having a cooling fluid.

FIGS. 17J and 17K illustrate an embodiment having a distal surface that is displaced in one direction along a device axis 1991 that is orthogonal to device axis 1997.

FIG. 18 depicts a method of removing an encroaching structure in an epidural space, according to an embodiment.

### DETAILED DESCRIPTION OF THE INVENTION

Reference will now be made in detail to various embodiments of the disclosed devices and methods, examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

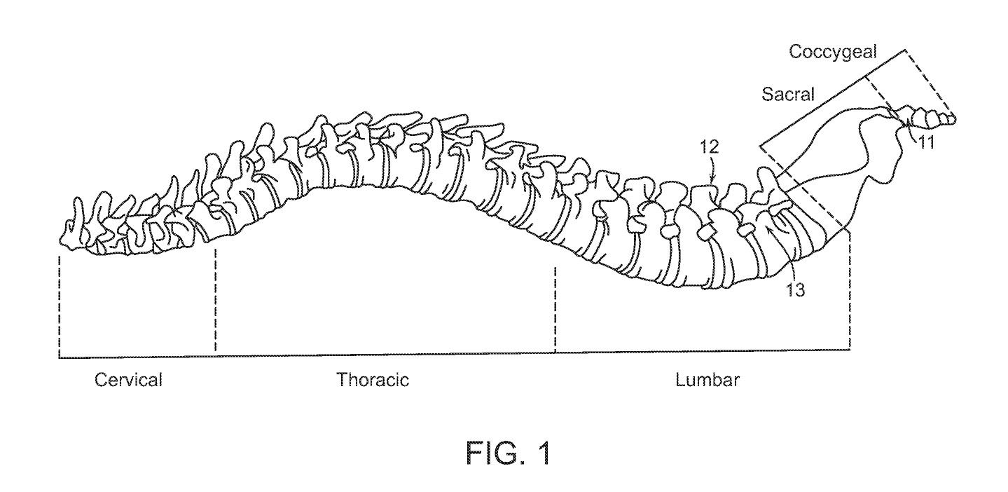

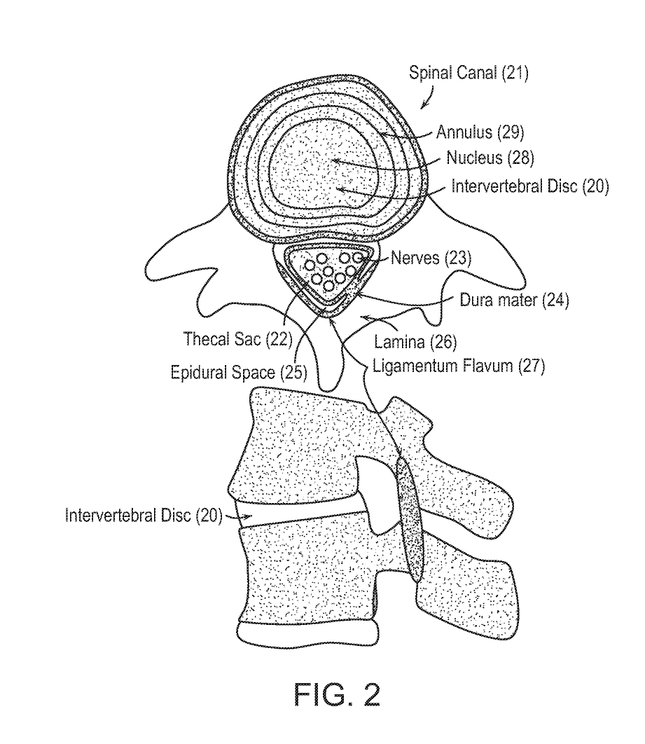

The basic structural unit of the spine is a vertebra. There are 7 individual vertebrae in the neck, 12 individual vertebrae in the upper and mid back (thoracic vertebra), and five individual vertebra in the lower back (lumbar vertebra) in the human spinal column. There are nine fused vertebrae below the lumbar vertebrae, namely, the sacrum (5 fused vertebrae), and the tail bone (4 fused vertebrae). The individual vertebrae are joined to each other in the front and the back. The structure of the human spine is shown in FIG. 1 and the structure of an individual vertebra with the intervertebral disc on top is shown in FIG. 2.

The spine has 33 vertebrae (7 cervical, 12 thoracic, 5 lumbar, 5 sacral, 4 coccygeal). Preferred embodiments of an endoscopic device can be advanced into all areas of the spinal canal and may be introduced from below, via the tail bone opening 11 (sacro-coccygeal hiatus), or the back, the interlaminar opening 12, or the side, the transforaminal opening 13. The device is flexible and preferably has a diameter of less than 5 mm in diameter. The cross sectional shape can be substantially circular, oval, rectangular, ellipsoidal or a non-uniform ellipse shape, depending upon the location of percutaneous entry. Generally, the cross-sectional area of the device is less than 20 mm², is preferably less than 14 mm², and to further reduce the risk of perforation of damage to the epidural membrane surrounding features is less than 10 mm².

The intervertebral disc is a cushion like structure in between the vertebrae that accommodates motion and absorbs shock as shown in FIG. 2 which schematically depicts a cross section of the spine. The intervertebral disc 20 is consequently a cushion between the vertebrae that enables freedom of movement while preserving the structure.

In the middle of the vertebra there is an opening or a hole, in the spinal canal 21. Inside the spinal canal 21 a fluid filled sac is positioned, the thecal sac 22. Inside the thecal sac 22 lie the nerves 23 and the spinal cord. The fluid in the thecal sac 23 is enclosed in a soft membrane, the duramater 24, which can be easily deformed.

The space between the duramater 24 and the bone of the vertebra is the epidural space 25. The epidural space 25 ends at the upper end of the spine and below at the base of the sacrum. Since the duramater 24 can be deformed, the epidural space 25 size may be increased manually by pushing with a mechanical device such as a balloon. The space created cannot, however, be sustained after the distending force dissipates given the outward force exerted on the duramater 24 by the fluid in the thecal sac 22.

The spinal canal 21 is a conduit for the spinal cord and the nerves 23. The margins of the spinal canal 21 are formed by the vertebral body or the disc in the front. In the back the margin is formed by the bony laminae 26 that are joined together by the ligamentum flavum 27. In the back the vertebra are attached to each other through several ligaments and joints. The ligamentum flavum 27 also connects the vertebrae in the back.

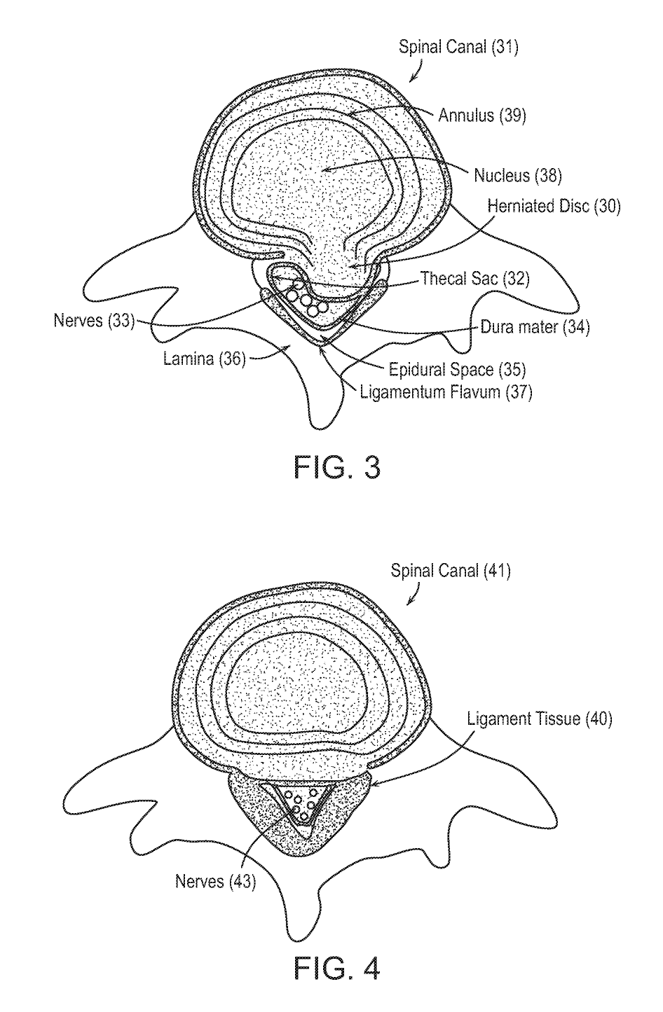

The intervertebral disc 20 is composed of a central soft gelatinous material in the center, the nucleus 38, which is enclosed in tough circumferential fibro cartilage, the disc annulus 39. With wear and tear, the disc annulus 39 may at times rupture allowing the disc to herniate or displace itself into the canal 31. FIG. 3 schematically depicts a disc herniation 30 that extends out of the disc center. The herniated disc 30 material creates severe pain because of irritation as well as pressure on the nerves and spinal cord that get impinged against the bony canal.

These may lead to neurological problems such as weakness, numbness as well as bladder and bowel incontinence. When the pain is severe or where it does not get better with time or where the patient develops neurological problems, the only option is that of removal of the herniated disc 30 requiring a significant surgical procedure by removing the bone and overlying tissue. This may also be amenable to endoscopic removal without removing the overlying bone.

The herniated disc 30 lies in the front part of the epidural space 35. As is seen in FIG. 3 the herniated material 30 is occupying at least a portion of the spinal canal 31 and creating pressure on the nerves 33. As is also clear from FIG. 3, to remove the disc, the overlying bone and tissue need to be removed surgically. However there is another option of threading a laser device to the area of the herniated disc material 30 and removing the herniated disc material 30 by vaporizing it, as the majority of the disc material 30 comprises water. The small amount of debris can be absorbed by the body or removed by suction, for example.

With aging the degenerative changes lead to bone overgrowth, soft issue overgrowth into the spinal canal 41 causing significant narrowing of the spinal canal. Some important causes of spinal stenosis include bone overgrowth, ligamentum overgrowth or disc herniation. FIG. 4 schematically depicts an example of spinal stenosis.

When there is significant narrowing of the size of the spinal canal 41 the important nerve 43 structures get pinched and their blood supply is compromised. This condition is called spinal stenosis. This leads to back and leg pain on standing and walking. If the symptoms are severe surgery is often required to create more space which is usually done by removing the bone in the back.

In FIG. 4, the cause of spinal stenosis is ligament tissue 40 overgrowth. It is clear from this figure that to remove the stenosis the overlying tissue 40 will need to be removed surgically. However there is another option of threading a laser device to the area and removing the overgrown tissue 40 and bone as necessary by vaporizing it. The small debris can be absorbed by the body. The innovation in this device specifically pertains to access of the stenotic area, visualization of the region and safe ablation of the tissue 40.

Various invasive surgical methods are used when there is encroachment upon the spinal canal. For a disc herniation a discectomy can be done. The disc and some overlying bone and tissue are removed. When there is significant surgery, additional screws and plates may also be placed to fuse the bones and maintain stability of the spine. In a further embodiment removal of a disc can also be done endoscopically through a rigid tube greater than 5 mm in diameter. Epidural scopes that are smaller than 5 mm can be used for ablation of a disc, however, poor visualization has prevented such procedures.

Various minimally invasive methods are also available for the treatment of spinal stenosis. Minimally invasive lumbar decompression involves manual removal of ligamentum flavum under X-Ray with rigid instruments, but is done without direct visualization.

Other methods depend on altering the biomechanics of the spine by opening up the space between the vertebra using rigid instruments such as the X-Stop® that is available from Paradigm Spine LLC, New York, N.Y. Another minimally invasive method for diagnosis and treatment of spine conditions via the epidural space is epiduroscopy using a device called the epiduroscope. The epidural space can also be accessed with a needle.

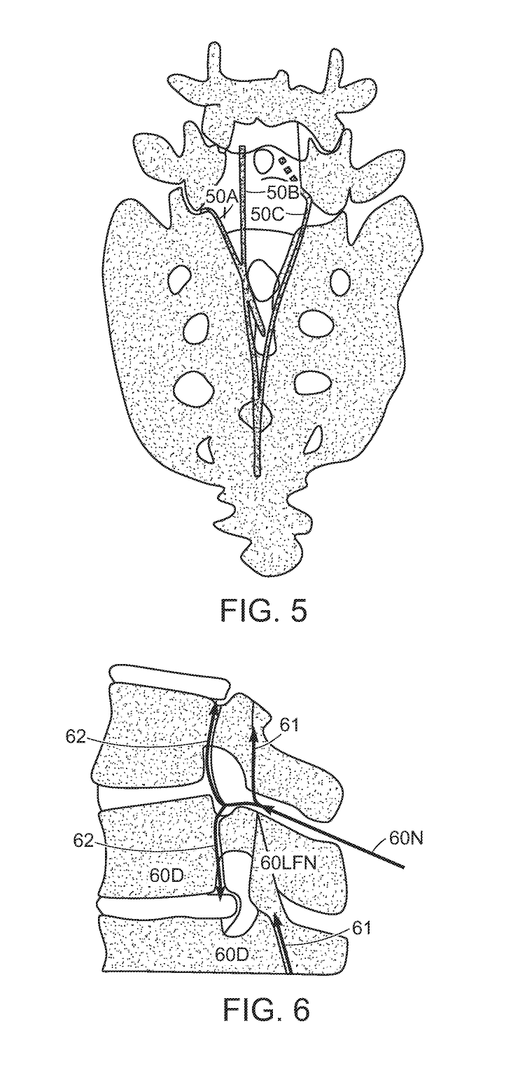

The epidural space ends at the sacrum. The last sacral bones fail to join in the midline leaving an opening covered by skin and soft tissue. A needle may be placed through this opening to enter the epidural space, “the caudal approach” as depicted in FIG. 5. This approach used for epiduroscopic procedures in accordance with preferred embodiments of the present invention.

For example, the epidural space and epiduroscopy may be performed by the sacral hiatus approach. Wire 50A is directed into the neural foramen for this method. Wire 50B can be directed posteriorly in the epidural space for the approach for ligamentum flavum resection for treatment of spinal steno sis. Wire 50C can be directed into the front and this approach is appropriate for removal of a herniated disc.

As an example, a needle is first inserted into the sacral hiatus at the opening at the bottom of the spine. The epidural space can be identified by loss of resistance technique. A wire can then be threaded into the epidural space. A curved wire is preferred to aid in navigation to the desired location. The semi-rigid wire (0.5 mm-2 mm) is slowly advanced by gentle direct force. The tip is directed to reach the correct compartment of the epidural space. The access device or a working channel may be then threaded over the wire to reach the area of pathology. In case of difficulty, the wire may expand by inflating like a balloon to dilate the track. Similarly dilators of different sizes may also be used sequentially to thread over the wire to create space for the access device or epiduroscope. The dilators are made of plastic or metal with variable rigidity and diameter. These may be threaded over the wire in a sequential fashion to create space for the epiduroscope when difficulty arises in threading the epiduroscope.

A probe with a working channel can be placed initially instead of the scope. Once it is threaded over the wire to the correct location the epiduroscope can slide into position within the working channel for example. Thus the diameter of the working channel would be such as to accommodate the access device or epiduroscope within it. The probe with the working channel can be flexible or semirigid with a soft rounded tip formed by a stylet placed within it. The soft rounded distal tip may be soft or semi rigid and can be shaped to facilitate displacement of the epidural membrane for a specific application. In another instance the tip of the stylet may be inflatable to dilate the tract, or distal region, within the epidural space when needed. The stylet can thus be used to initially deflect the membrane adjacent to the structure to be ablated and thereby enable visualization and treatment. As the distal end of the stylet is retracted into the working channel, the distal tip of the working channel can be translated to maintain separation of the membrane from the adjoining structure. In a further embodiment, the access device has individual lumous for imaging, laser light delivery, illumination suction, coolant flow, fluid delivery and component may be placed in the lumous individually as needed during a surgical procedure.

The epidural space may be accessed with a needle placed in the interlaminar space and thus going between the bones, referred to herein as “the interlaminar approach” as depicted in FIG. 6. For example, the wire is inserted into the epidural space from the back. It may then be advanced in the posterior compartment 61 of the epidural space (appropriate approach for spinal stenosis), or in the anterior compartment 62 (appropriate approach for disc herniation). The dotted line is the duramater 60D, the shaded area is the hypertrophied ligament 60LFH and the needle is represented by letter 60N.

As another example, the epidural space can be reached by threading a needle from the back. The needle may be straight or curved. The epidural space is identified by loss of resistance technique. A wire may then be threaded into the epidural space. A curved wire is optimal. The semi-rigid wire (0.5-2 mm) is slowly advanced by gentle direct force. The tip is directed to reach the correct compartment of the epidural space. The tip may be directed towards the head or the foot based upon where the narrowed area to be treated is located. The device or the epiduroscope can then threaded over the wire to reach the area of pathology. In case of difficulty, the wire may expand by inflating like a balloon to dilate the track. Similarly, dilators of different sizes may also be used sequentially to thread over the wire to create space for the device.

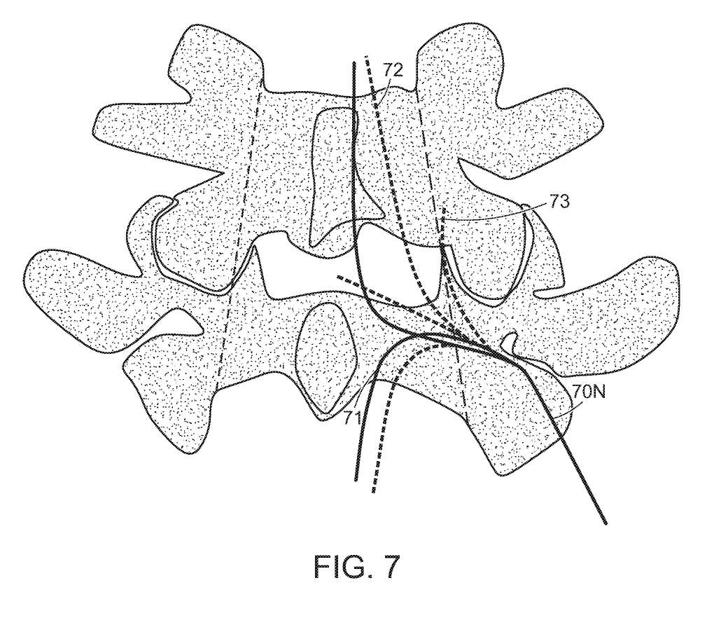

On the side of the spine is an opening called the intervertebral foramen. A needle may be placed directly in the epidural space through this opening, the “transforaminal approach” as depicted in FIG. 7 which is a view from the back of the spine. The curved needle 70N is placed into the foramen from the side and the wire (thick line) is advanced into the posterior epidural space 71, appropriate for ligamentum decompression or the anterior epidural space, dotted line 72 appropriate for disc herniation or the foramen, and the lateral epidural space 73 appropriate for disc herniation and foraminal decompression.

As an example, a curved needle is placed into the side opening in the spine where the nerves emerge known as the intervertebral foramen. Once the needle is placed a wire is threaded into the epidural space. Needle adjustment may be needed until the wire can be threaded. A wire may then be threaded into the epidural space. A curved wire is optimal. The semi-rigid wire (1-2 mm) is slowly advanced by gentle direct force. The tip is directed to reach the correct compartment of the epidural space. The tip may be directed towards the head or the foot based upon where the narrowed area to be treated is located. The device or a working channel may be then threaded over the wire to reach the area of pathology. In case of difficulty, the wire may expand by inflating like a balloon to dilate the track.

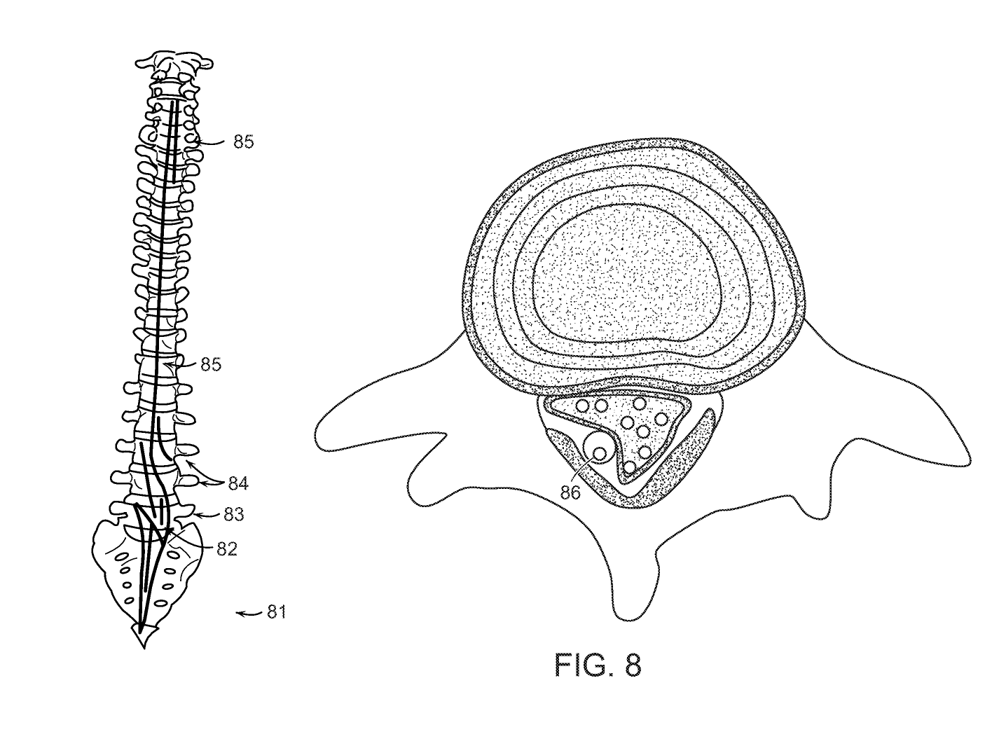

FIG. 8 schematically depicts various methods of entering the epidural space superimposed over a spine model. 81 represents sacral hiatus insertion, 82 represents sacral hiatus to foramen, 83 represents sacral hiatus to epidural space, 84 represents transforaminal approach to epidural space, 85 represents an interlaminar approach, 86 is a diagram of an epiduroscope in the dorsal epidural space reflecting the duramater.

During epiduroscopy semi rigid or flexible tubing with an inbuilt camera and a working channel may be used for diagnosing and treating spine conditions. It is most frequently used for removing adhesions that may form after spine surgery. The diagnostic and therapeutic utility of the method is limited and is not currently considered a part of standard treatment algorithm and is employed infrequently. The problems relating to visualization and safe ablation continue to be problematic.

The present invention is described and made to deliver a flexible device by the interlaminar, transforaminal, or sacral route to the area of encroaching pathology in conditions such as spinal stenosis and disc herniation. The instruments and method of accessing the area of pathology is described. The instruments and method for visualizing the pathology are described. The instrument and method to ensure safety are described. The instrument and methods to remove the pathology are described. The device will help realize the promise of minimally invasive surgery of the spine by solving the problems of visualization and safety while also realizing effective decompression.

The present invention is designed to access the area of pathology in all areas of the spine, including the back and the neck. The device may be placed into the epidural space using interlaminar (back), caudal (tail bone) or transforaminal (side) approach at any level of the lumbar, thoracic and cervical spine. The device may be advanced in the anterior (front) or lateral (side) or epidural posterior (back) epidural space for pathology such as disc herniation or spinal stenosis.

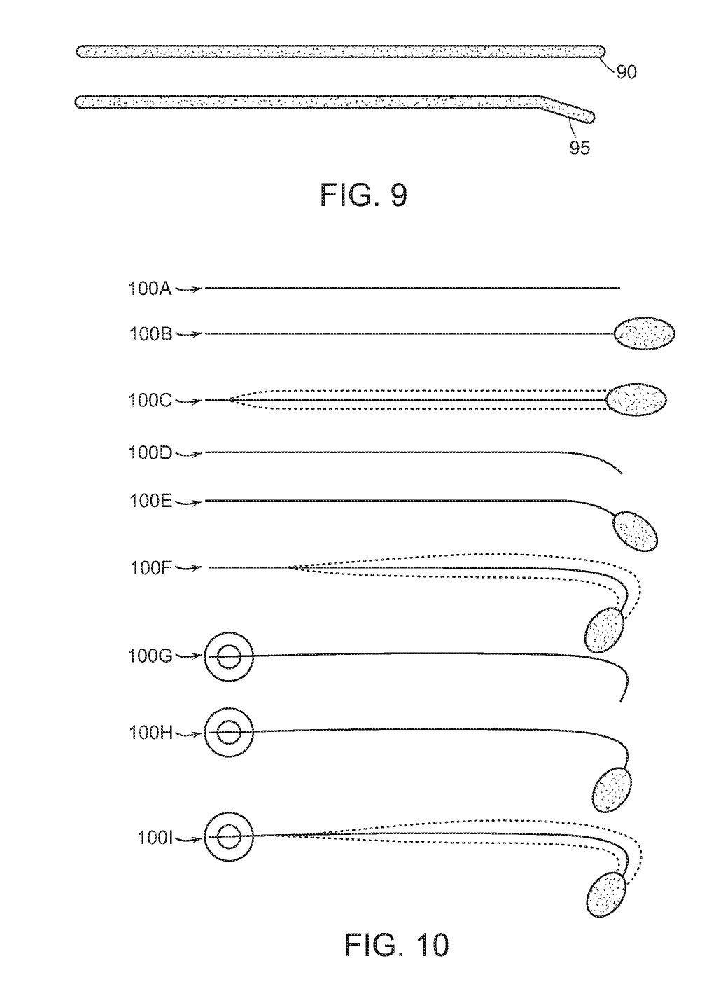

The epidural space may be accessed with a straight or curved needle by the interlaminar, transforaminal, or sacral route. FIG. 9 schematically depicts a straight needle 90 and a curved needle 95 according to an embodiment. Metallic needles may be used. The needles can be hollow to enable introduction of flexible tubular bodies.

A wire may then be threaded through the needle tip. FIG. 10 schematically depicts a wire according to multiple embodiments. Wires 100A-C represent straight wire. Wires 100D-F represent curved wires prebent or with a hollow core in which a curved stylet may be placed and appropriately curved. Wires 100G-I represent wires in which the curvature may be increased or decreased using a plurality of joints.

Wires 100A, 100D, 100G have no dilation tools. Wires 100B, 100E, and 100H have a dilation tool at the tip such as an inflatable balloon tip or a balloon may be advanced through the wire core and inflated at the tip. The tip may be wrapped in an inflatable membrane that can be inflated from outside. For wires 100C, 100F, 100I the entire wire or portions of thereof may be inflatable aiding the dilation of the space for allowing an epiduroscope or access device to pass. The wire may be wrapped in an inflatable membrane that can be inflated from outside. The membrane may have compartments allowing for segmental inflation. The wire may be solid or with hollow core. The wire may have straight or curved tip. The curved wire can assist in controlling wire tip motion. The curve may be attained by using a curved stylet or a pre-bent wire. By using the curve the wire may be advanced into the intended area under X-Ray or other methods of control such as ultrasound, or other neuro-navigation tools.

The wires range from 0.5-2 mm, may be hollow or solid, and are made from metal or plastic. Curved wires may have a pre-bent tip or a hollow core into which a curved stylet may then be introduced. The wire may also have an adjustable curve, via a plurality of joints to allow for precise navigation in the epidural space. The epidural wires may also have an inflatable balloon tip to allow for creation of space when there is difficulty in navigation and to avoid puncturing the dura. The tip itself may be covered by an inflatable membrane or a balloon may be introduced through the hollow core for this purpose. The entire wire or parts of the wire may be covered by an inflatable membrane to allow for dilation of the epidural space and for easy passage of the epiduroscope or the access device.

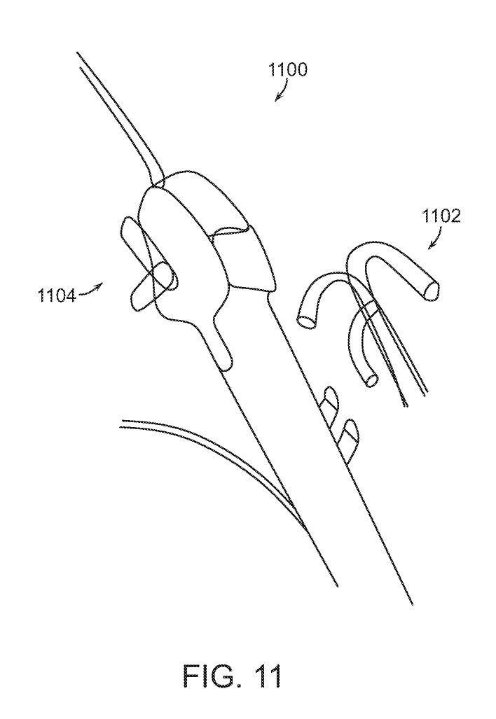

FIG. 11 schematically depicts a tip of a working channel device or epiduroscope 1100 according to a preferred embodiments. Note that the access device can comprise an epiduroscope or a combination with other components as described herein. In some embodiments, the wire tip curvature 1102 may be variable and controlled by the operator for greater precision. The curvature may be modulated by employing a plurality of joints. In some embodiments, the device tip can have a balloon to distend the area if unable to navigate. In some embodiments, device tip can have a stylet to form smooth passage for the device. A balloon or membrane can be inflated to distend the passage to enable visualization. The tip 1104 of the working channel device may be rotated or bent in different planes using wires that lie in the body of scope and are attached to the tip.

The sub 5 mm device with a stowed expandable tip may then be advanced over the wire. In another embodiment, the device tip can be non-expandable. The tip of the device may be moved in 1 or 2 or multiple planes employing a plurality of joints. The tip of the device has metallic strips interspersed with transparent plastic.

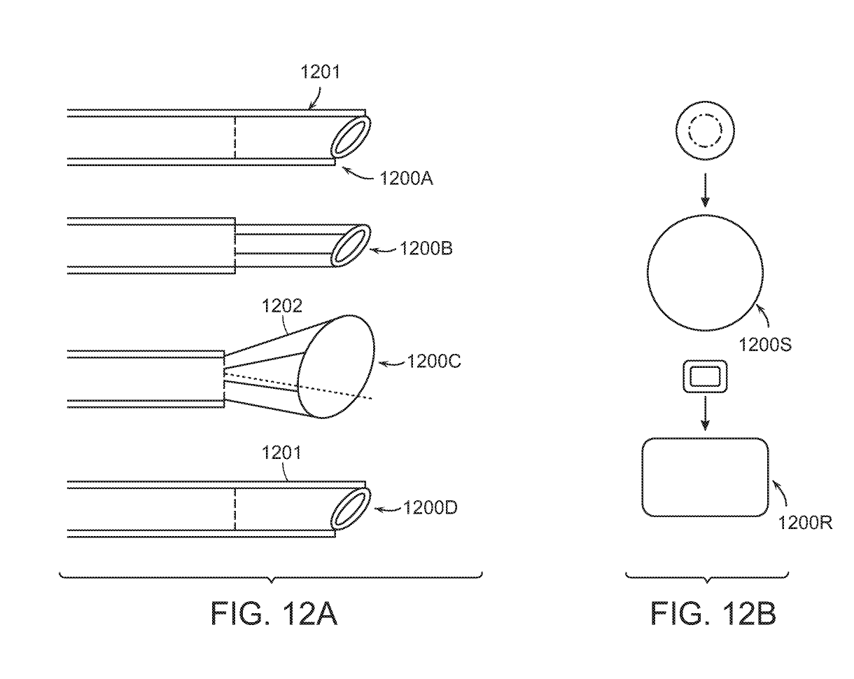

FIG. 12A schematically depicts unstowing and stowing of the tip of the device according to an embodiment. The tip of the device is covered circumferentially by a sleeve at the tip only or a circumferential outer tube throughout the length of the scope. When in the appropriate area, the sleeve or the outer tube is withdrawn to allow deploying of the tip. After exposure, the tip of the device can be unstowed by using a plurality of joints. The tip may be again stowed by advancing the outer tube or sleeve back over it.

The tip is covered by a sleeve 1201 that may extend the entire length of the device or just at the tip 1200A. The inner tube tip is in a stowed position. The outer sleeve is withdrawn using a trigger or other actuation mechanism in the handle of the scope 1200B. The tip may then be unstowed or dispensed for use. In this particular instance the tip is unstowed by tugging on the wires 1202 attached to the tip 1200C. Note that only the dural half of the tip can be moveable. This is accomplished by pulling a lever in the handle. The tip may be unfurled into a spherical configuration 1200S or more of a rectangular configuration 1200R as seen in FIG. 12B. The tip can be beveled (or not beveled) and the longer edge is optionally color coded red, for example. The device tip can be non-beveled and the dural edge, that is, the tip portion that engages and moves the durameter can be identified with a radio-opague marker. The forward facing laser beam centered at a point that can be (or not be) offset towards the receding edge. Other portions of the tip can also be radio-opaque to provide for appropriate recognition under fluoroscopy. The outer tube or sleeve maybe advanced back over the tip to stow it and the scope navigated into the other area as needed. In a preferred embodiments, only a portion of the distal circumference serves to displace the membrane to form a three dimensional visualization and ablation space. The various embodiments herein can incorporate this feature. Note that the base of the access device or epiduroscope can be enclosed or covered with an inflatable membrane to enable controlled movement in the anterior posterior plane.

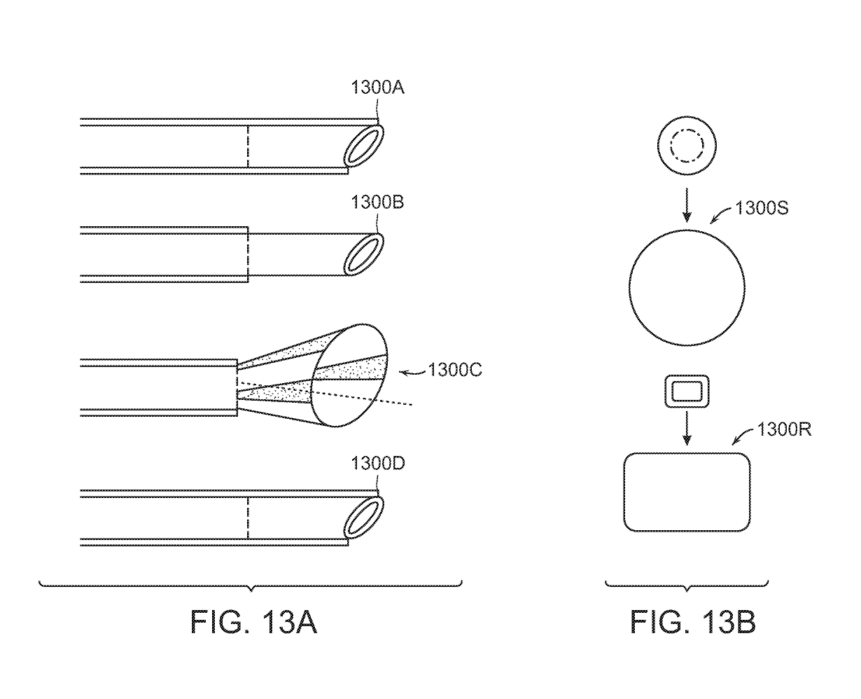

FIG. 13A schematically depicts unstowing and stowing of the tip 1300A-1300D analogous to 1200A-1200D of the epiduroscope when the tip 1300C is made of nitinol in an expanded shape according to an embodiment. In this embodiment, the tip is made of metal strips such as nitinol in an expanded shape. This is kept stowed by the outer tube which can be moved back and forth to enable rapid stowing and unstowing. As the outer tube is withdrawn, the inner stowed tip unfurls or moves into the preformed shape. As depicted in FIG. 13B in circular 1300S rectangular 1300R or other shape described herein. The outer sleeve or tube slides back on and the tip is again stowed allowing for further endoscope motion.

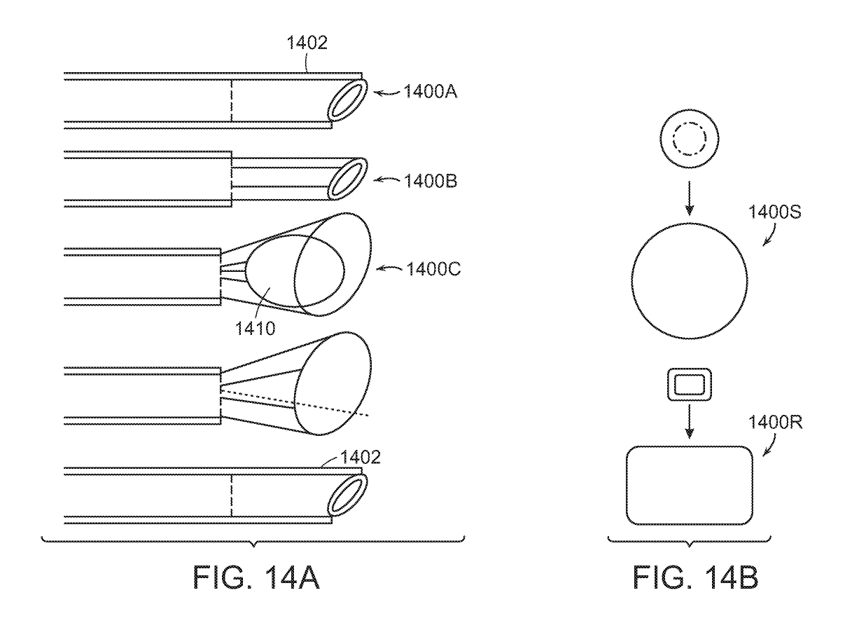

FIG. 14A schematically depicts unstowing and stowing of the tip of the device when the tip is unstowed with a balloon according to an embodiment. After exposure, the tip 1400C of the epiduroscope may be unstowed by deploying a balloon 1410 that is inserted through the working channel. The tip may be again stowed by advancing the outer tube or sleeve back over it. The metal strips or wires in the tip prevent spontaneous collapse. The sleeve or outer tube 1402 may be moved backwards and forwards using a lever at the proximal end and thus accomplishing opening and closing of the unstowed tip. Once the tip is stowed the scope can be mobilized in the epidural space again. The shape of the deployed structure is illustrated in FIG. 14B.

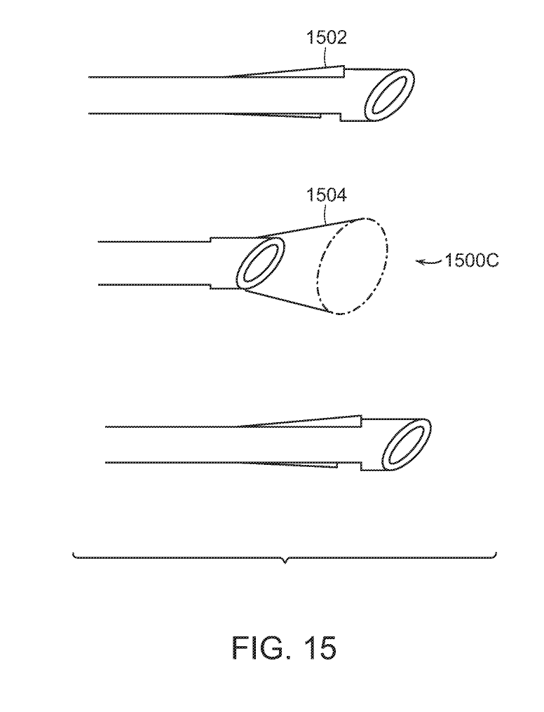

FIG. 15 schematically depicts unstowing and stowing of the tip 1500C of the device when a stowable sheath 1502 is behind and external to the tip according to an embodiment. The stowable sheath 1502 is behind and external to the distal end of the scope. As the tip 1500C is retracted this deploys the sheath. As the tip is advanced the sheath is stowed. The distal edge of the outer tube overlies the proximal edge of the inner tube. As the inner tube is withdrawn it flays metal strips 1504 on the outer tube creating a funnel or rectangular shaped shield that displaces the dura mater allowing greater visualization through the inner tube. The inner tube is pushed distally collapsing the shield. The tip maintains shape from natural plasticity of inserted metallic strips.

The scope has a proximal and distal end. The proximal end can have a lever allowing motion of the tip through inbuilt control wires. The handle has an intake for the light source, video output from the CMOS or CCD sensor, or fiberoptic channel, a port for a laser, and two working channels. The laser channel is adaptable to all lasers suitable for the ablation procedure but a preferred embodiment utilizes CO₂ laser delivery. Such lasers can operate at a wavelength of 10,600 nm and have output powers in a range of 40-100 watts that can be operated in a pulsed mode using pulse width modulation. The scope may be of variable length based upon the particular application such as whether to be used in the back or the neck. In some embodiments, the scope has a width of approximately in a range of 3-7 mm, and preferably at about 5 mm or less.

The tubular body can possess varying degrees of flexibility. The body can be a double tube or a single tube with a coaxial external tip at the distal end. The outer tube the distal tip can be retractable to deploy and stow the expandable tip. The handle has levers for mobility of the distal tip in one or more planes.

A balloon can be advanced through the working channel and inflated at the tip to allow for smooth distal tip to allow navigation and decrease risk of dural puncture. In embodiments where the expandable tip is activated by wires there is a lever for stowing and retracting the tip as well as a lever for sliding the sleeve or the outer tube. In embodiments where the expandable tip is composed of a material with metal memory such as nitinol, there is a lever in the handle for activating the outer sleeve or tube of the scope that leads to opening and closing on the tip. In embodiments where the expandable tip is activated by balloon, a balloon can slide through the working channel and expand the tip. A lever retracts the outer tube. The outer tube can slide back over the expanded tip to be stowed. In embodiments the outer tube or the distal tip can have a hinged end such that pulling the inner tube deploys the outer tube or outer distal tip to allow visualization in this manner.

The stowable tip is slightly oblong with the longer side color coded and directed to the dura mater. At the distal end of the tip is the laser beam aperture, two working channels and a camera sensor. The forward facing laser can be directed slightly off center towards the closer portion of the target material.

In an embodiment, only the access channel device is initially introduced. It has the stowable tip that can be deployed using any of the methods described above. Once it is deployed the device with a laser emission port, one or two working channels and a camera can then be introduced to reach the distal end of the stowable tip of the working channel.

In an embodiment, the epiduroscope or access device can be semi-rigid or rigid with a flexible tip. In some embodiments, the access device or the cannula and stowable tip can be tubular structure or have of a substantially rectangular or ellipsoid cross section or profile.

In an embodiment a wire with an inflatable hood may be advanced through the working channel and advanced over the intruding pathology providing a safety wall to the spinal sac distally. In an embodiment, a color shield or balloon may be introduced from the opposite side to provide a barrier and an end point to the firing laser.

The distance of the laser tip from the pathology is radiologically and visually ascertained. A measuring tool such as a rigid wire or rod can be advanced from the distal tip to contact the pathology or tissue/material to be removed. This can include a sensor that indicates the distance to the target region, computes and indicates the spot size and communicates the power requirements and can automatically set the illumination parameters. The laser can be operated in a continuous mode, a pulsed mode, or super pulsed mode. When the disc is at an optimal distance from the pathology and the safe side of the cannula is placed toward the bone the CO₂ laser is fired. The laser emission is adjusted based upon radiological measures so that damage is restricted to the target and not beyond. The laser is fired under continuous visual monitoring. The tip is stowed and the cannula moved slowly as and when needed. Saline flush or other fluid or gas flow can optionally be used for removal of the vaporized tissue. The ablation process continues until all the area of pathology is ablated visually as well as determining the status radiologically by x-ray, ultrasound or computed tomography (CT) imaging procedures. A wire can also be extended to form the distal tip for distance measurement.

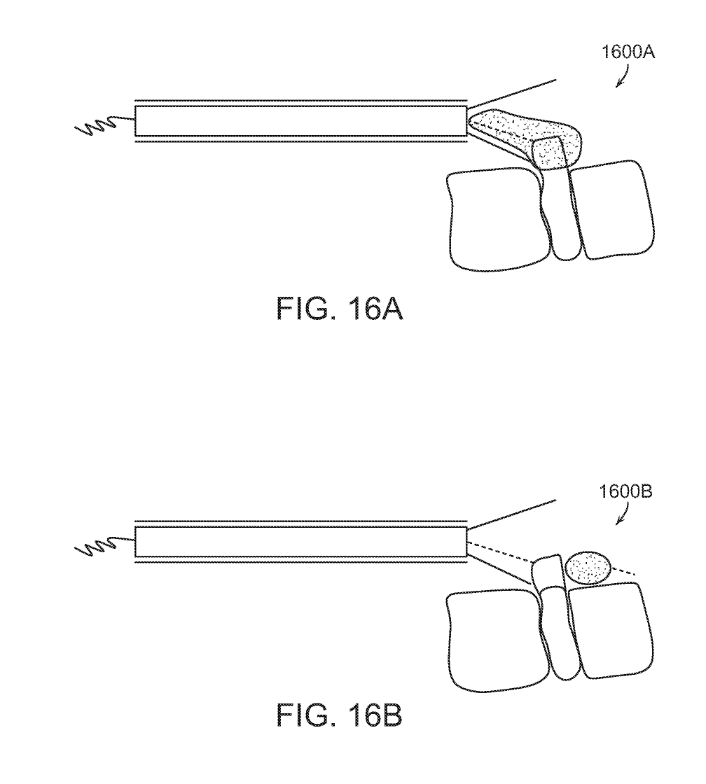

FIG. 16A schematically depicts a balloon 1600A distally advanced over a herniated disc according to an embodiment. This provides a distal shield as well as indicator for procedure completion. FIG. 16B schematically depicts a balloon 1600B proximally advanced over a herniated disc according to an embodiment.

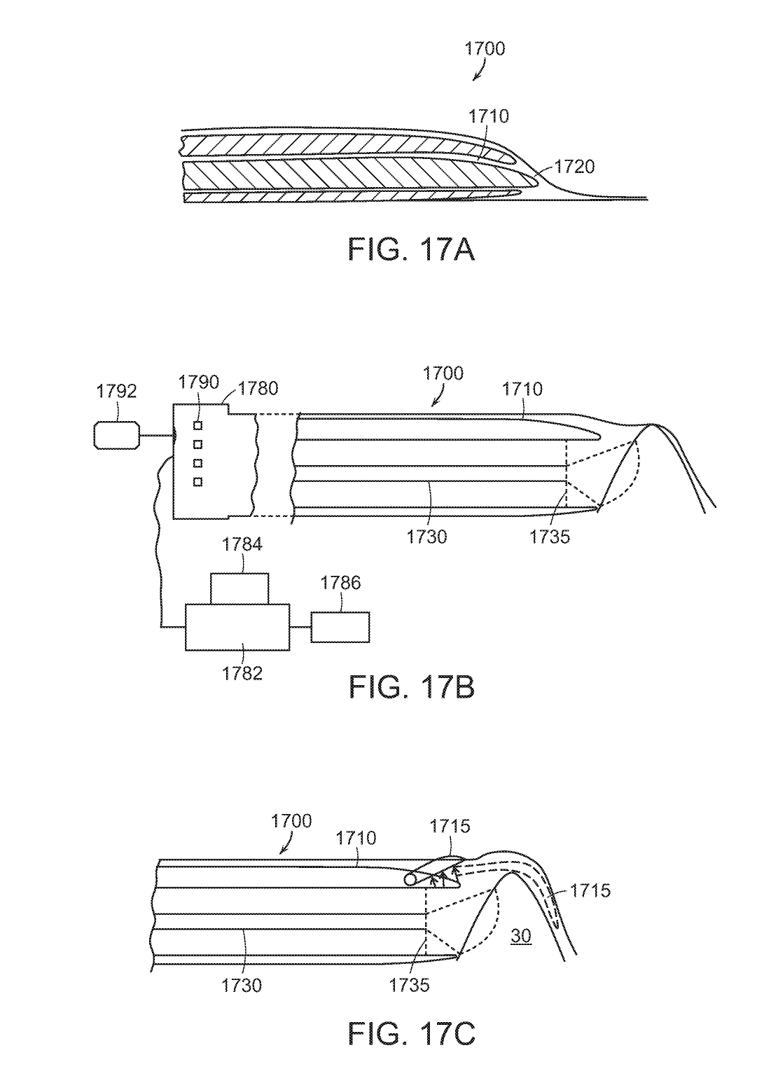

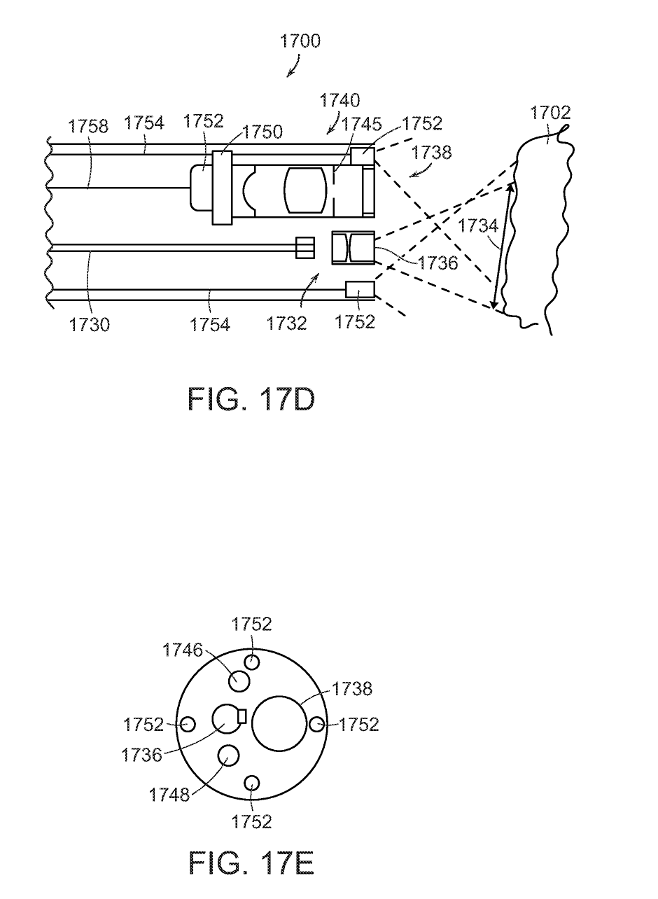

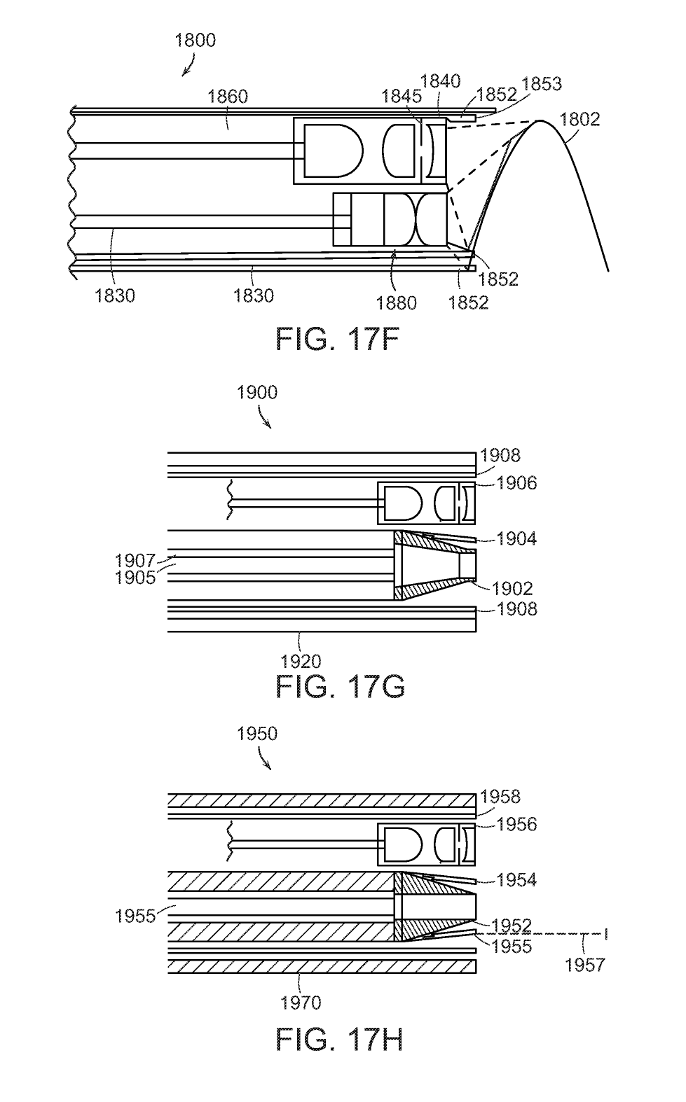

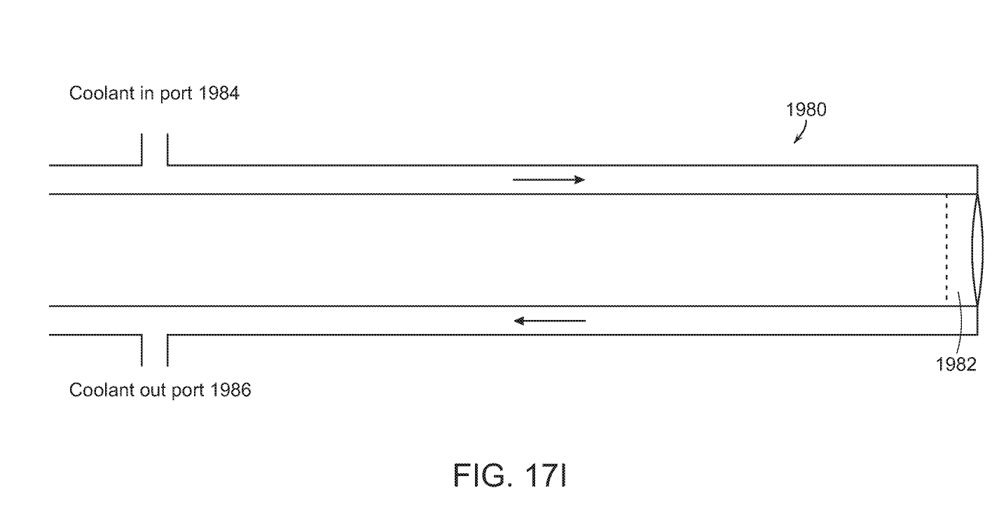

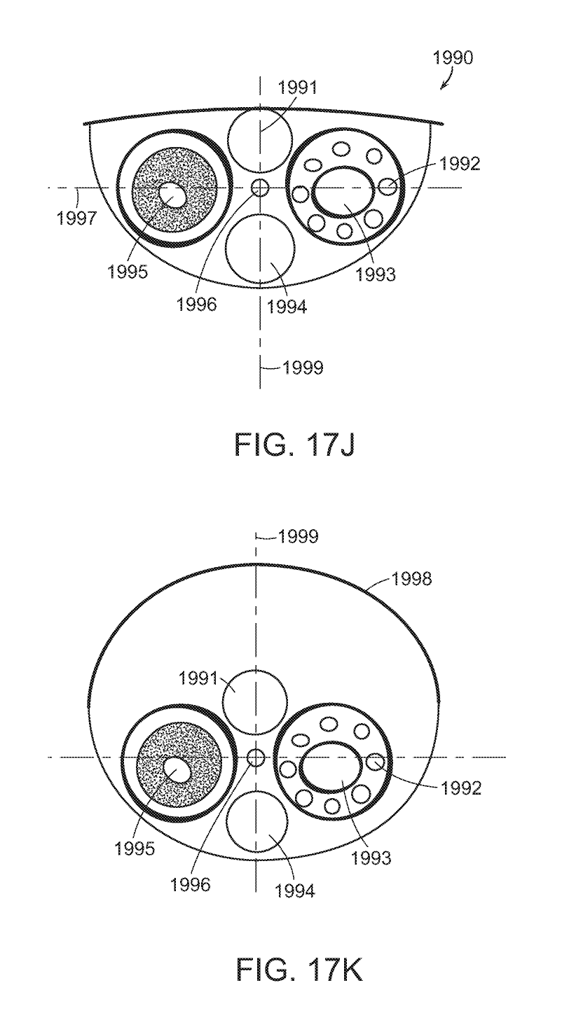

FIG. 17A schematically depicts a stylet for introducing an epiduroscope 1700 moving in an epidural space between a membrane and a spine according to an embodiment. The epiduroscope comprises a working channel 1710. Within the working channel 1710 is a stylet 1720. The working channel 1710 is shaped to smoothly move under membranous tissue without tearing the membrane. Additionally, the shape of the working channel 1710 allows for extension and withdrawal of the stylet 1720 without inhibiting the movement of the stylet 1720. The distal tip of the stylet 1720 is marked, dyed, coated, or otherwise manufactured to be visible via x-ray, fluoroscopy, or other diagnostic tool. In some embodiments, the distal tip may be fluorescent. The distal tip of the stylet 1720 is a domed, blunt nose that deflects the membrane around the sides of the tubular body of the stylet 1720. Note that the distal tip of the stylet can include an inflatable device or membrane to gently form a cavity in case of an obstruction, scar tissue or adhesions.

Fig, 17B schematically depicts an epiduroscope 1700 approaching an intruding pathology according to an embodiment. Upon reaching the pathology, the stylet 1720 can be withdrawn. In some embodiments, the stylet 1720 is withdrawn by twisting the stylet 1720 and pulling the stylet 1720 away from the pathology. A laser 1730 is then positioned at a predetermined position 1735 within the working channel 1710. Specifically, the laser 1730 is positioned in a position 1735 at a distance from the pathology wherein the laser 1730 is able to ablate the pathology.

FIG. 17C schematically depicts an epiduroscope 1700 with a moving member attached to a distal end such as a hinged member 1715 according to an embodiment. As the stylet 1720 is withdrawn, a hinged member 1715 on the distal end of the working channel 1710 moves forward to maintain an opening to view and treat the surgical site. The hinged member 1715 maintains an opening for the 1730 to ablate the intruding pathology. The member 1715 can also extend to the opposite side of the material 30 such that the light that is used to ablate tissue 30 does not fully penetrate and thereby damage the epidural membrane. Alternatively, a second probe can be percutaneously inserted from a different location on the opposite side of tissue 30 that is positioned to absorb or reflect light from the laser that would otherwise be directed onto the inner surface of the membrane or other adjacent tissue. Note that the devices described herein can include handle enabling manual manipulation and actuation of electronic components. The handle 1780 can be connected to computer 1782, image display 1784, and data storage 1786 devices. The handle 1780 can have manual actuators 1790 to trigger the laser that is located in the handle or in an external laser light source such as CO₂ laser 1792 which can be coupled into the handle by rigid waveguides, flexible hollow fiber as described herein or free space lens assembly, imaging devices, LED illumination, target illumination, and distance measuring element. The handle can include imaging sensors coupled to fiber optics, light sources for illumination and a control processor connectable to an external computer and network.

FIGS. 17D-17G depict embodiments that can be inserted into the working channel of FIG. 17A or can be used as integral units having the dilating elements described generally herein. FIG. 17D schematically depicts a distal end of a preferred epiduroscope including an optical fiber or bundle of optical fibers 1730 to deliver light onto an area 1734 of tissue 1702 to be removed. The lenses 1740, aperture stop 1745 and a digital imager 1750 of the epiduroscope 1700 enable visualization of the tissue 1702. After withdrawing the stylet 1720, for example, the tissue visualization and ablation device is inserted through the working channel 1710. The surgeon first illuminates the tissue to visually identify the area to be ablated and positions the device to direct a beam of light onto a spot 1734 of a tissue surface. The lenses 1740 receive light from the field of view, including the tissue 1702 to be removed. The resulting image is detected by the digital imager 1750. In some embodiments, the digital imager 1750 is a CMOS sensor. In some embodiments, the digital imager 1750 is a charge-coupled device (CCD). The digital imager 1750 preferably has at least 50,000 pixels and preferably 300,000 pixels for high resolution imaging at video frame rates. A small diameter imaging device such as that described in U.S. application Ser. No. 15/051,265 filed on Feb. 23, 2016 can be used, the entire contents of the above application being incorporated herein by reference. The digital imager 1750 transmits a digital image data to a computer for processing and display. The imaging device can include a processor 1752 that processes the image data and transmits the data through conductive connector or wire to the proximal end of the endoscope. As seen in FIG. 17E, the distal ends of the fibers or the LEDs 1752 can be arranged in an annular array within the tubular body 1747 to more evenly illuminate the field of view. The imaging aperture 1738 and the light emission aperture 1736 for the removal of tissue are preferably aligned upon a central axis of the tubular body 1700. The field of view can be illuminated by one or more light emitters 1752 which can be optical fibers, optical fiber bundles or light emitting diodes (LEDs) mounted at the distal end of the endoscope. The visualization and ablation device can optionally include a suction channel 1746, and a fluid delivery channel 1748 an instrument channel or a balloon sheath.

FIG. 17F schematically depicts an optical fiber 1830 to deliver light for ablation of tissue in which, lenses 1880, evenly distribute the light onto tissue 1802. A fiber optic imaging channel 1860 of an epiduroscope according to this embodiment couples the image to an image sensor at the proximal end. The lenses 1840 can be optically coupled to a distal end of a fiber optic imaging channel that can extend through the working channel 1710 to enable viewing of the region of interest. The fiber optic imaging channel 1860 preferably has at least 300,000 pixels, and preferably more than 1 million pixels for high resolution imaging at video frame rates. The fiber optic imaging channel 1860 transmits the image that is delivered to a detector which generates digital image data to a computer for processing and display.

As shown in FIGS. 17D and 17F, the digital imager 1750 or the fiber optic imaging channel 1860 can be mounted within a second tubular body in which a laser light delivery system can also be mounted such that the digital imager 1750 or the fiber optic imaging channel 1860 and related optical elements are arranged to view the illuminated region of tissue. A second white light source such as a light emitting device (LED) can be used to provide illumination of the small surgical field of view.

Preferred embodiments of the invention relate to the use of light sources emitting at wavelengths that will ablate or vaporize tissue to be removed from a surgical site for treatment of spinal injury or conditions that impair movement and/or cause pain. A CO₂ laser can be used to emit a beam of light that is coupled into a waveguide of an endoscope or epiduroscope for delivery to a location within the epidural space.

In FIG. 17G, a probe body 1920 has been inserted into the epidural space. The body 1920 can be a flexible tubular member that is inserted into the epidural space using procedure described in the present application. Light from a CO₂ laser is coupled into waveguide 1907 to couple light into distal beam shaping element 1902 waveguide 1907 can be flexible and use silica and silver layers such as those available from Laser Engineering Inc. in Milford, Mass.. An optical fiber 1904 can be used to illuminate the spot that is to be ablated by the light emitted from element 1902. Note that a hard ceramic can be used at the distal tip of the waveguide. Fluid aspiration can be used to clean the tip during the procedure. The spot generated by optical fiber 1904 is pre-aligned with the spot illuminated by element 1902 so that a user can see the tissue region to be ablated by one or more laser pulses emitted from the distal aperture of element 1902. Visualization channel 1906 is positioned for viewing tissue to be ablated at a distance from the distal end of the device. Annular light emitting elements 1908 are used to illuminate the entire field of view to enable steering of the targeting light spot provided by fiber 1904. Alternatively, a beam of visible light can also be coupled into channel 1905 of waveguide 1907 using a mirror and a further LED light source. FIG. 17H shows a second beam shaping element 1952 that projects a spot having a different size onto the defect to be treated. The emission aperture can be open and can use a fluid such as a gas flow to maintain the waveguide free of body fluids or debris. Alternatively the emission aperture can be covered or enclosed with a light transmissive cap or window at the wavelengths of the CO₂ laser. A probe element 1955 can be extended distally to contact material to be ablated and thereby determine a distance 1957 from a distal end of the device in the present and previously described embodiments. This distance can be used to automatically compute ablation laser parameters based on spot size.

As shown in FIG. 17I a coolant can be introduced into the catheter or endoscope body 1980 where fluid is directed through port 1984 into one or more channels within the tubular body towards the distal end where in passes along a distal channel 1982 and reverses direction to exit port 1986. This embodiment can be used for application using continuous wave or longer pulse duration applications to ablate material for certain applications.

FIGS. 17J and 17K illustrate cross-sectional views of an embodiment having a first curved side 1990 that expands to a larger diameter 1998 to move the epidural membrane and thereby form the visualization and ablation of cavity.

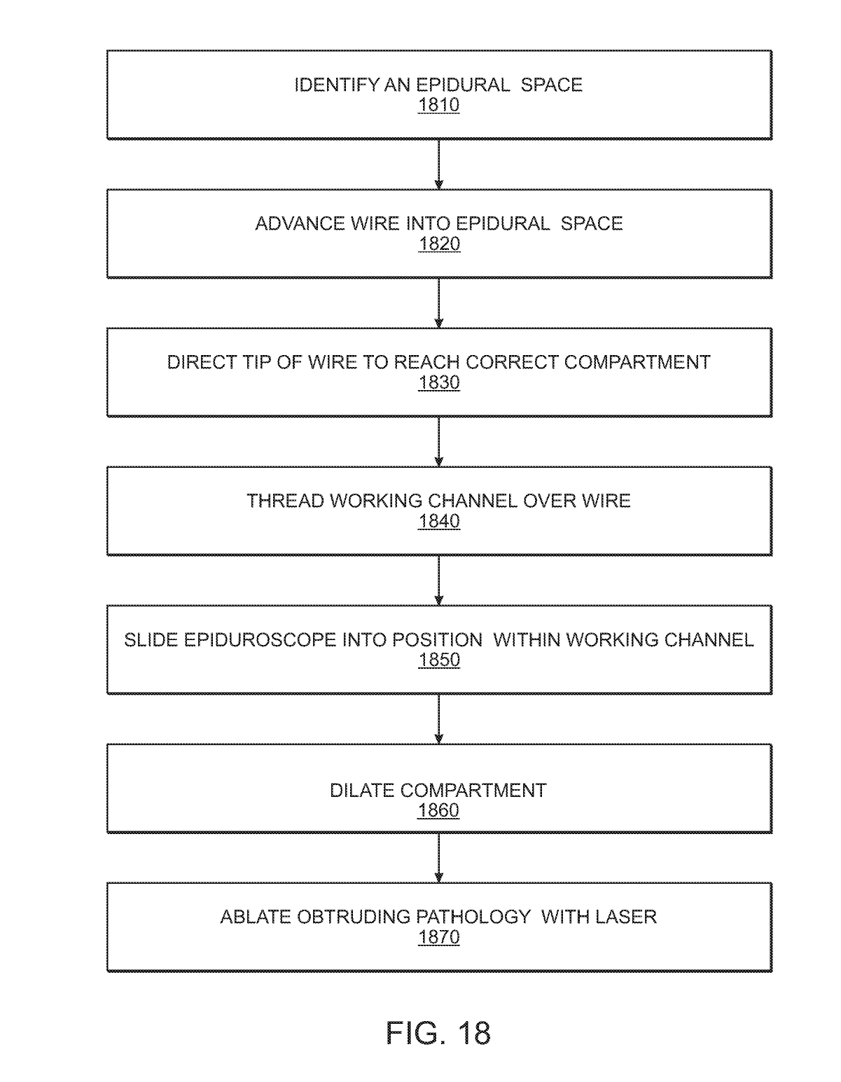

FIG. 18 depicts a method of removing an encroaching structure in an epidural space, according to an embodiment. The method begins when an epidural space is identified (Step 1810). The epidural space can be identified by loss of resistance technique. A wire is advanced into an epidural space (Step 1820). A curved wire is preferred to aid in navigation to the desired location. The wire may be semi-rigid (0.5-2 mm) and is slowly advanced by gentle direct force.

The tip of the wire is then directed to reach the correct compartment (Step 1830). A working channel is threaded over the wire (Step 1840). Once the working channel is threaded over the wire to the correct location an epiduroscope is slid into position within the working channel (Step 1850). If necessary, the compartment is dilated (Step 1860). Dilators of different sizes may also be used sequentially to thread over the wire to create space for the epiduroscope. The dilators are made of plastic or metal with variable rigidity and diameter. These may be threaded over the wire in a sequential fashion to create space for the epiduroscope when difficulty arises in threading the epiduroscope.

Then, intruding pathology is ablated with a laser (Step 1870). The laser may be a CO₂ laser. In an embodiment a wire with an inflatable hood may be advanced through the working channel and advanced over the intruding pathology providing a safety wall to the spinal sac distally. In an embodiment, a color shield or balloon may be introduced from the opposite side to provide a barrier and an end point to the firing laser.

Other embodiments will be apparent to those skilled in the art from consideration of the specification and practice of this disclosure. It is intended that the specification and examples be considered as exemplary only, with the true scope and spirit of the disclosed devices and methods being indicated by the following claims.

1. A device for surgical treatment of a spinal defect, comprising:
a handle attached to a tubular body including at least one channel, the tubular body having a size that can be inserted into an epidural space along a spine of a patient;
a laser optically coupled to an optical fiber device extending within the tubular body such that emitted light is transmitted in a distal direction to ablate tissue associated with a spinal defect;
an illumination device that emits light at the distal end of the tubular body to illuminate a field of view; and
an imaging device to image a body region distal to the tubular body to be treated with the emitted light.

2. The device of claim 1 further comprises a dilator that displaces tissue within the epidural space.

3. The device of claim 1 further comprising a sleeve retractably covering at least a distal end of a first wire, wherein the sleeve is retracted upon actuation of an actuator in the handle.

4. The device of claim 1 further comprising a first wire threaded through a distal end of a needle wherein an inflatable hood or membrane is configured to displace tissue.

5. The device of claim 1 further comprising a second wire threaded through a distal end of the needle, wherein a distal end of the second wire comprises an inflatable hood advanced over an intruding pathology to provide a barrier between the laser output and surrounding tissue.

6. The device of claim 1 wherein a balloon can be inserted through a proximal opening in the hand.

7. The device of claim 1 wherein the laser comprises a carbon dioxide laser.

8. The device of claim 7 further comprising a guide light source that illuminates a region of tissue such that a user can identify the region of tissue for ablation.

9. The device of claim 1 wherein the laser comprises a light emitting diode that emits one or more wavelengths within an infrared range.

10. The device of claim 1 wherein the laser comprises a Nd:YAG laser or a Ho:YAG laser that emits at one or more wavelengths in a range of 800 nm to 2000 nm.

11. The device of claim 2 wherein the dilator comprises a balloon or a moveable element mounted on a distal end of the tubular body.

12. A method for treating a spinal defect comprising:
introducing a tubular body into an epidural space adjacent to a spinal region;
positioning a distal end of the device to view of tissue in the epidural space;
illuminating the defect with light emitted from a distal end of the tubular body to visualize the defect; and
directing laser light onto the defect to remove at least a portion of the tissue from the epidural space.

13. The method of claim 12 further comprising emitting light using a carbon dioxide laser and coupling the emitted light into a waveguide comprising a hollow fiber body.

14. The method of claim 12 further comprising removing at least a portion of tissue positioned in the epidural space from a herniated disc material.

15. The method of claim 12 further comprising removing at least a portion of tissue positioned in the epidural space from a spinal stenosis.

16. The method of claim 12 wherein introducing the tubular body into the spinal region comprises percutaneously inserting the tubular body with an interlaminar approach, a transforaminal approach or a sacral hiatus approach.

17. The method of claim 12 further comprising distending the epidural space with a distal end of the tubular body.

18. The method of claim 12 further comprising detecting light from the epidural space with a detector and displaying an image.

19. The method of claim 16 wherein the tubular body has a curved distal surface that displaces an epidural member that covers the epidural space, the distal surface being percutaneously introduced through skin of a patient and further comprising displacing the distal surface along a length of the spinal region.

20. The method of claim 17 further comprising distending an epidural membrane with a balloon or a displaceable member.
